SEQUENCE LISTING (27 sequences)

SEQ ID NO: 1 - DNA, 16 bp, Artificial Sequence
  Synthetic construct
tacgggaggcagcagt

SEQ ID NO: 2 - DNA, 55 bp, Artificial Sequence
  Synthetic construct
aattctaatacgactcactataggggctgctggcacgtagttagccgtggctttc

SEQ ID NO: 3 - DNA, 31 bp, Artificial Sequence
  Synthetic construct
cgagctagcaacgccgcgtgagtgaagctcg

SEQ ID NO: 4 - DNA, 186 bp, Artificial Sequence
  Synthetic construct
tacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagcccc

SEQ ID NO: 5 - DNA, 22 bp, Artificial Sequence
  Synthetic construct
cctgatgcagccatgccgcgtg

SEQ ID NO: 6 - DNA, 45 bp, Artificial Sequence
  Synthetic construct
aattctaatacgactcactatagggcacggagttagccggtgctt

SEQ ID NO: 7 - DNA, 37 bp, Artificial Sequence
  Synthetic construct
cgagcttgaagaaggccttcgggttgtaaagagctcg

SEQ ID NO: 8 - DNA, 38 bp, Artificial Sequence
  Synthetic construct
tgaaagtactttacaacccgaaggccttcttcatacac

SEQ ID NO: 9 - DNA, 132 bp, Artificial Sequence
  Synthetic construct
cctgatgcagccatgccgcgtgtatgaagaaggccttcgggttgtaaagtactttcagcggggaggaagggagtaagttaatacctttgctcattgacgttacccgcagaagaagcaccggctaactccgtg

SEQ ID NO: 10 - DNA, 20 bp, Artificial Sequence
  Synthetic construct
ggaatccgctaaggagtgtg

SEQ ID NO: 11 - DNA, 46 bp, Artificial Sequence
  Synthetic construct
aattctaatacgactcactatagggccatccattttcagggctagt

SEQ ID NO: 12 - DNA, 32 bp, Artificial Sequence
  Synthetic construct
cgcgattaacaactcaccggccgaatatcgcg

SEQ ID NO: 13 - DNA, 33 bp, Artificial Sequence
  Synthetic construct
gctagttcattcggccggtgagttgttacacac

SEQ ID NO: 14 - DNA, 63 bp, Artificial Sequence
  Synthetic construct
ggaatccgctaaggagtgtgtaacaactcaccggccgaatgaactagccctgaaaatggatgg

SEQ ID NO: 15 - DNA, 20 bp, Artificial Sequence
  Synthetic construct
cagcagttggacatgggtta

SEQ ID NO: 16 - DNA, 45 bp, Artificial Sequence
  Synthetic construct
aattctaatacgactcactataggggagaatccacatccaggtgc

SEQ ID NO: 17 - DNA, 30 bp, Artificial Sequence
  Synthetic construct
cgaccggcatagggaagttccgtttggtcg

SEQ ID NO: 18 - DNA, 33 bp, Artificial Sequence
  Synthetic construct
cgcctttcaaacggaacttccctatgccttagg

SEQ ID NO: 19 - DNA, 123 bp, Artificial Sequence
  Synthetic construct
cagcagttggacatgggttagtcgatcctaaggcatagggaagttccgtttgaaaggcgccctcgtgcgccgtgtgccgaaagggaagccggttaacattccggcacctggatgtggattctc

SEQ ID NO: 20 - DNA, 20 bp, Artificial Sequence
  Synthetic construct
cggctcttcctatcataccg

SEQ ID NO: 21 - DNA, 45 bp, Artificial Sequence
  Synthetic construct
aattctaatacgactcactatagggctaaacccagctcacgttcc

SEQ ID NO: 22 - DNA, 32 bp, Artificial Sequence
  Synthetic construct
cgcgatattcggtaagcgttggattgatcgcg

SEQ ID NO: 23 - DNA, 33 bp, Artificial Sequence
  Synthetic construct
tgggtgaacaatccaacgcttaccgaattctgc

SEQ ID NO: 24 - DNA, 82 bp, Artificial Sequence
  Synthetic construct
cggctcttcctatcataccgaagcagaattcggtaagcgttggattgttcacccactaatagggaacgtgagctgggtttag

SEQ ID NO: 25 - DNA, 32 bp, Artificial Sequence
  Synthetic construct
gaccttcatcactcacgcggcgttgctccgtc

SEQ ID NO: 26 - DNA, 65 bp, Artificial Sequence
  Synthetic construct
ttttttttttaattctaatacgactcactataggggctgctggcacgtagttagccgtggctttc

SEQ ID NO: 27 - DNA, 26 bp, Artificial Sequence
  Synthetic construct
tttttttttttacgggaggcagcagt

(19) United States
(12) Patent Application Publication
(10) Pub. No.: US 2016/0115535 A1
(43) Pub. Date: Apr. 28, 2016

(54) Gel-Tethering for Integrated Oligonucleotide Amplification and Real-Time Detection

(71) Applicant: THE TRUSTEES OF THE STEVENS INSTITUTE OF TECHNOLOGY, Hoboken, NJ (US)

(72) Inventors: Xiaoguang Dai, Weehawken, NJ (US); Youlong Ma, Hoboken, NJ (US); Matthew Libera, New Providence, NJ (US)

(73) Assignee: The Trustees Of The Stevens Institute Of Technology, Hoboken, NJ (US)

(21) Appl. No.: 14/605,013

(22) Filed: Jan. 26, 2015

Related U.S. Application Data
(60) Provisional application No. 61/932,559, filed on Jan. 28, 2014.

(51) Int. Cl. C12Q 1/68

(52) U.S. Cl. CPC C12Q 1/6876

(57) ABSTRACT

A device for detecting biospecific oligonucleotides includes a plurality of microgel spots, each of which is functionalized with molecular beacon probes and amplification primers tethered thereto. Each of the respective probes is arranged to bind to an antisense counterpart of one type of biospecific oligonucleotide. The various microgels may each be functionalized for a different oligonucleotide. In a system that incorporates the aforesaid device, the device is in contact with a solution that includes a system of molecules that cooperated with the tethered probes and primers to capture, amplify, and detect the antisense counterparts of one or more biospecific oligonucleotides. In one such system, the enzymes and primers are arranged to implement a NASBA amplification process operating on the biospecific oligonucleotides.

### CROSS-REFERENCE TO RELATED APPLICATION

The present application claims the benefit of U.S. Provisional Patent Application No. 61/932,559, filed on Jan. 28, 2014, which is incorporated by reference herein in its entirety.

### STATEMENT REGARDING FEDERALLY SPONSORED RESEARCH

This project was partially supported by the U.S. Army Research Office via Grant No. W911NF-12-0331 and by the National Science Foundation via Grant No. IIP 1262903 and Grant No. CBET-1402706. The Government of the United States of America may have certain rights with regard to the disclosed subject matter.

### FIELD OF THE INVENTION OR TECHNICAL FIELD

This invention relates to the field of biochemical analysis, more specifically, the use of patterned surfaces and microarrays for detection and quantification of biochemical targets, such as those associated with oligonucleotide detection and analysis.

### BACKGROUND OF THE INVENTION

The effective treatment of an infection requires microbial identification in order to prescribe an appropriate antimicrobial treatment. The traditional diagnostic approach cultures blood, sputum, urine, washes, swabs, or other bodily fluids to determine if microbes are present, and then examines phenotypes within the culture, such as morphology or antimicrobial susceptibility, for identification. This approach is slow. It often requires periods as long as 3-5 days to complete, during which time the prescription of antibiotics is under-informed and fraught with problems including serious threats to patient well-being.

Molecular diagnostics (MDx) have radically changed the process of clinical microbial identification (see, Deshpande et al., Expert Review of Molecular Diagnostics, 2012. 12(6): pp. 645-659; Millar et al., Current Issues in Molecular Biology, 2007. 9(1): pp. 21-40; and Muldrew, Current Opinion in Pediatrics, 2009. 21(1): pp. 102-111, the disclosures of all three of which are incorporated by reference herein). Importantly, MDx approaches are both specific and fast. They can identify microbes to the species and strain level based on biospecific markers over a time scale that can be as short as one hour. Consequently, clinicians can administer the most effective and appropriate antimicrobial treatment at an early time point with substantial implications both for patient well-being and for easing the burden on the health-care system.

Surface-patterned microgels provide a fundamentally different platform for the design and development of clinically relevant molecular diagnostics. Microgels can be localized on a surface in an array format with feature (spot) sizes as small as a few hundred nanometers, and they can be synthesized with reactive groups, such as biotin, which enable their functionalization with oligonucleotides or other biomolecules. Importantly, and unlike the vast majority of other surface-tethering methods, surface-patterned microgels not only physically separate the oligonucleotides from the underlying solid surface, but, because of the highly hydrated structure at the microgel surface, they tether the oligonucleotides in an environment much like the surrounding aqueous medium. This preserves their biospecific properties. Furthermore, in contrast to approaches that entrap probes (see, Livshits and Mirzabekov, Biophysical Journal, 1996. 71(5): pp. 2795-2801; Proudnikov et al., Analytical Biochemistry, 1998. 259(1): pp. 34-41; Rehman et al., Nucleic Acids Research, 1999. 27(2): pp. 649-655; Rendl et al., Langmuir, 2011. 27(10): pp. 6116-6123; and Rubina et al., Analytical Biochemistry, 2004. 325(1): pp. 92-106, the disclosures of all five of which are incorporated by reference herein) or primers within a hydrogel, microgel tethering positions the oligonucleotides at or near the gel surface rather than within the gel itself, thus providing maximum accessibility for hybridization and complexation.

While there are many possible ways to detect a hybridization event, fluorescence is among the most common. This is a simple mechanism that can be detected and processed using simple optics and electronics. The fluorescence-detection process can be further simplified by using self-reporting hybridization probes known as molecular beacons (see, Tyagi, S. and F. R. Kramer, Nature Biotechnology, 1996. 14(3): pp. 303-308, the disclosure of which is incorporated by reference herein) or other similar self-reporting probes. Molecular beacon probes fluoresce when hybridized to a complementary target, and they thus eliminate the additional need to label the target prior to, during, or after, hybridization to the probe. They furthermore enable real-time detection of the signal during, rather than after, an experiment or test, because the signal is emitted once a hybridization event occurs. Invented in the mid-1990's, they have been extensively used in basic scientific studies of cellular and sub-cellular processes, and they have played a key role in a number of highly successful commercial ventures associated with sequencing or detection (see, Tyagi, S. and F. R. Kramer, F1000 Medicine Reports, 2012. 4(1), the disclosure of which is incorporated by reference herein).

Despite their many successes, molecular beacon probes have thus far been used almost exclusively untethered in solution. While they can be immobilized on solid surfaces by several mechanisms (see, Du et al., J Am Chem Soc, 2005. 127(21): pp. 7932-40; Du et al., J Am Chem Soc, 2003. 125(14): pp. 4012-13; Liu et al., Analytical Biochemistry, 2000. 283(1): pp. 56-63; Martinez et al., Anal Chem, 2009. 81(9): pp. 3448-54; Situma et al., Analytical Biochemistry, 2007. 363(1): pp. 35-45; Song et al., Angew Chem Int Ed Engl, 2009. 48(46): pp. 8670-4; Stoermer et al., J Am Chem Soc, 2006. 128(51): pp. 16892-903, the disclosures of all seven of which are incorporated by reference herein), their performance when tethered has typically been much poorer than that when free in solution. The background fluorescence associated with surface-tethered beacons has traditionally been high, so the signal above background is low. This phenomenon has been attributed to the fact the beacon's probe sequence, fluorophore, or quencher can nonspecifically interact with the substrate to produce background fluorescence without a biospecific hybridization event. Consequently, molecular beacon probes have not been extensively used in microarray formats.

The problem of preserving high signal-to-background when tethering molecular beacon probes to a surface has been addressed (see, Dai et al., Soft Matter, 2012. 8(11): pp. 3067-3076; Dai et al., WO/2013/089888, the disclosures of both of which are incorporated by reference herein) by tethering the beacons to highly hydrated microgels patterned by a focused electron beam on a glass or Si substrate (see, Wang et al., J Polymer Science, Part B: Polymer Physics, 2013, the disclosure of which is incorporated by reference herein). This important gel-tethering approach brings at least two significant advantages. First, the beacons are grafted to the outermost surface of the microgel. In contrast, trapping oligonucleotide probes within a gel not only restricts the possible conformations of the various biomolecules but also slows the reaction kinetics because of the need for diffusion through tortuous pathways within the gel prior to binding. Both of these constraints are eliminated when the probes are tethered to the surface of the gel. Second, the nature of the e-beam patterned microgels is such that the crosslink density at the outer surface gradually approaches zero, so the probes that are tethered at the outer surface find themselves extremely unconstrained and in the most water-like environment possible. Hence, their performance can approach that of untethered beacons in aqueous solution where the probes are unrestricted.

Microgels can be particles of any shape whose equivalent diameter is approximately 0.1-100 μm. Surface-pattern microgels can be created by techniques of photolithography (see, Revzin et al., Langmuir, 2001. 17(18) 5440-5447, the disclosure of which is incorporated by reference herein). However, e-beam patterning of microgels brings particular flexibility to the creation of the surface-patterned microgels for use in self-reporting, multiplexed, microarrays needed to interrogate a physiological sample such as blood with a large set of questions. Unlike spotting methods that create individual array spots with diameters of approximately 100-500 μm (see, Rubina et al., Analytical Biochemistry, 2004, 325(1), 92-106, the disclosure of which is incorporated by reference herein), e-beam patterning can create discrete microarray spots with diameters ranging from sub-micron (one microgel) to tens/hundreds of microns (multiple overlapping microgels). Individual array spots can be positioned in user-defined shapes, at specific locations on a surface, at controllable distances from each other, and in any number of patterns, including 1-D or 2-D arrays within a microfluidic channel or a chamber. Furthermore, discrete spots within the array can be differentially functionalized. For example, functionalization at the scale of approximately 1-10 μm can be achieved using a method such as dip-pen nanolithography or at the scale of approximately 10-100 μm using a position-sensitive microspotting robot.

Molecular beacons can be tethered to a microgel by a number of chemistries. Among the successful chemistries is the biotin-streptavidin interaction. Microgels can be synthesized using biotinylated PEG (B-PEG) homopolymer and then activated by exposure to streptavidin. Biotinylated oligonucleotide(s) can subsequently be deposited onto the activated microgel spot(s) by one of the micro or nano-spotting methods. For microgels e-beam synthesized by e-beam under typical conditions (e.g., 100 nm films of 5 kDa B-PEG homopolymer and 50 fC of 2 keV focused electrons), each microgel can be created so that it presents about 11,000 active binding sites (see, Dai et al., Soft Matter, 2012. 8(11): pp. 3067-3076; Dai et al., WO/2013/089888, the disclosures of which are incorporated by reference herein).

Because the concentration of targets (e.g., microbial RNA) in a specimen such as blood is typically low, amplification is used to generate detectable signals. So-called target amplification creates copies of the target RNA (or DNA). PCR is one-such amplification method. It has been extensively used in a variety of formats. Nucleic Acid Sequence Based Amplification (NASBA) is another established but less-common method (see, Deiman et al., Applied Biochemistry and Biotechnology—Part B Molecular Biotechnology, 2002. 20(2): pp. 163-179, the disclosure of which is incorporated by reference herein). It directly amplifies RNA, and, unlike RT-PCR, is not susceptible to contamination by genomic DNA. Furthermore, NASBA is isothermal, and a technology based on NASBA thus eliminates the added complexity of thermal cycling required for PCR. Many different amplification methods are available including, for example, Transcription-Mediated Amplification (TMA), Strand Displacement Amplification (SDA) and many variations of PCR.

In the art, the NASBA process has been implemented with the reagents dissolved in one or more solutions. The NASBA process uses three enzymes: Avian Myeloblastosis Virus Reverse Transcriptase (AMV-RT); RNase H; and T7 RNA Polymerase (T7 Poly). It also uses two primers: the P2 forward primer and the P1 reverse primer. In a solution-based NASBA process, these reagents are mixed with nucleic acids and strain-specific RNA (target RNA+) isolated from target microbes in the physiological sample. Using strain-specific target RNA+ isolated during sample preparation, the initiation phase produces ds-DNA templates with a T7 promoter region from which the target antisense RNA (RNA-) is produced. The cyclic phase uses this target RNA− to make additional ds-DNA from which more copies of RNA− amplicons are created at an increasing rate.

Multiplexing imposes important challenges to an amplification process. When probing for multiple target microbes in a single reaction chamber, primer sets needed to amplify each particular target must be in that chamber. As the number of possible targets increases, the number of primers and their overall concentration increases. When the primers are untethered and free in solution, they can complex to form, for example, primer-dimers rendering them ineffective for the amplification process. When performing a multiplexed amplification process in a single solution, the amplification efficiency thus decreases as the degree of multiplexing increases. One method of circumventing this problem is simply to create individual reaction chambers, each of which contains only one primer set and the corresponding probe, and all of which contain a sample of the parent target-containing solution. Such an approach has been used successfully in commercial PCR-based systems.

### SUMMARY OF THE INVENTION

Embodiments of the present invention provide gel-tethered molecular beacon probes and corresponding amplification primers, microarrays that include discrete regions each with biospecific combinations of gel-tethered probes and primers, and related methods of analysis and detection of multiple biological targets using such probes, primers and arrays.

In one aspect, the present invention provides a device for detecting multiple biological targets (e.g., nucleic acids or peptides). Embodiments of the device include a plurality of quenched molecular beacon probes capable of binding in a complementary manner to molecules that are complementary to the probes, a plurality of amplification primers, and an array of spatially-separated structures on a solid surface. The conformational changes in the probes that take place during binding cause the probes to fluoresce. In embodiments of the present invention, the complementary molecules are uniquely identified with the target molecules in such a way that the fluorescence of the probe signals the presence of the biological target.

In a second aspect, the present invention provides a conjugate for amplifying and detecting a target. Embodiments of the conjugate have: (i) a gel having a diffuse surface; (ii) a molecular beacon probe that is linked to the gel; and (iii) one or more amplification primers that are linked to the same gel.

In a third aspect of the present invention, the aforementioned device, one or more conjugates, or an array described above is used in a method for detecting the presence of one or more targets in a biological sample. Embodiments of methods according to the present invention include the steps of (a) providing a device, one or more conjugates, or an array described above; (b) contacting the device, the one or more conjugates, or the array with the biological sample for a period of time under conditions permitting binding between the one or more targets and the corresponding molecular beacon/hairpin probes or to the corresponding amplification primers; and (c) determining the presence of the binding between the one or more targets and the corresponding probes thereby detecting the presence of the one or more targets.

A fourth aspect of the present invention provides methods for making a conjugate described above. Methods according to the present invention include the steps of: (i) providing a gel that contains a first member of an affinity pair; (ii) contacting the gel with molecular beacon probes specific for a target, each molecular beacon probe comprising a second member of the affinity pair, for a period of time under conditions permitting binding between the first member and second member; (iii) removing molecular beacon probes that do not bind to the first member; (iv) contacting the gel with amplification primers that produce targets specific to the molecular beacon probes, each primer comprising another member of the affinity pair, for a period of time under conditions permitting binding between the first member and second member; and (v) removing primers that do not bind to the first member.

A fifth aspect of the present invention provides methods for making an array, such as the arrays discussed above. Such methods include the steps of: (1) obtaining a plurality of conjugates described above, which are specific for a plurality of targets, respectively; (ii) obtaining a support that has a plurality of unique locations; and (iii) depositing said plurality of conjugates on the plurality of unique locations, respectively.

### BRIEF DESCRIPTION OF FIGURES

For a better understanding of the present invention, reference is made to the following detailed description of the exemplary embodiments considered in conjunction with the accompanying drawings, in which:

FIG. 1 is a schematic illustration of a generalized molecular beacon probe and its target;

FIGS. 2A-2C constitute a schematic illustration of the synthesis and properties of surface-patterned microgels created by electron-beam patterning of biotinylated PEG thin films, according to an embodiment of the present invention;

FIG. 3 is a schematic illustration of the NASBA amplification process as conducted according to an embodiment of the present invention;

FIGS. 4A and 4B constitute a schematic illustration of the NASBA process of FIG. 3 in a solution which contacts microgel-tethered molecular beacon probes in a microarray spot on a solid substrate, according to an embodiment of the present invention;

FIGS. 5A and 5B constitute a schematic illustration of a partial gel-tethered NASBA implementation according to an embodiment of the present invention, with both primer P1 and the molecular beacon probes micro-gel tethered to a microarray spot and with the other reagents in the surrounding solution;

FIGS. 6A-6D constitute a schematic illustration of a hybrid process according to an embodiment of the present invention that includes amplification in the solution, amplification in conjunction with a gel-tethered primer, and detection by gel-tethered molecular beacon probes;

FIGS. 7A and 7B constitute a schematic illustration of the generation and hybridization of gel-tethered double strand DNA and gel-tethered molecular beacon probes according to an embodiment of the present invention;

FIG. 8 is a chart of the average fluorescence intensity emitted by three different microgel spots treated under three different protocols, according to embodiments of the present invention; and

FIGS. 9A-9E constitute a schematic illustration of gel-tethered amplification and gel-tethered real-time detection of a target RNA+, according to an embodiment of the present invention.

### DETAILED DESCRIPTION OF THE INVENTION

Embodiments of the present invention provide gel-tethered molecular beacon probes and corresponding amplification primers, microarrays that include discrete regions each with biospecific combinations of gel-tethered probes and primers, and related methods of analysis and detection of multiple biological targets using such probes, primers and arrays.

In one aspect, the present invention provides a device for detecting multiple biological targets (e.g., nucleic acids or peptides). Embodiments of the device include a plurality of quenched molecular beacon probes capable of binding in a complementary manner to molecules that are complementary to the probes, a plurality of amplification primers, and an array of spatially-separated structures on a solid surface. The conformational changes in the probes that take place during binding cause the probes to fluoresce. In embodiments of the present invention, the complementary molecules are uniquely identified with the target molecules in such a way that the fluorescence of the probe signals the presence of the biological target. In embodiments of the device, each structure contains a member selected from the group consisting of hydrogels, microgels, hydrogel coatings and hydrogel-like coatings, each having chemically or physically incorporated therein a linking moiety. The structure may be a surface-patterned microgel (e.g., an electron-beam surfaced patterned microgel). The microgel may comprise poly(ethylene glycol) or other polymers from which gels can be formed. In some embodiments, the structure is biotinylated, e-beam patterned poly(ethylene glycol) microgel.

In a second aspect, the present invention provides a conjugate for amplifying and detecting a target. Embodiments of the conjugate have: (i) a gel having a diffuse surface; (ii) a molecular beacon probe that is linked to the gel; and (iii) one or more amplification primers that are linked to the same gel. The molecular beacon probe is specific for the target. The amplification primers are specific to produce that same target. In embodiments of the present invention, the linkage may be a covalent or non-covalent chemical bond. In one embodiment, the molecular beacon probe and the primers are linked to the gel via an affinity pair. In embodiments of the present invention, the members of each affinity pair may be in a pair format (e.g., biotin-streptavidin). In embodiments of the present invention, the members of each affinity pair may be in a sandwich format (e.g., biotin-streptavidin-biotin). In embodiments of the present invention, the molecular beacon probe and primers are linked to a biotin, and the gel presents at or near its surface, biotin or streptavidin so that probe and gel are linked via the biotin-streptavidin pair. In other embodiments, the probe and corresponding primers are linked to the gel by a covalent pathway provided that the chemistry of the linking is orthogonal to other functional groups on the conjugate and on the probe.

In some embodiments of the present invention, the conjugate includes poly(ethylene glycol), poly(ethylene oxide), or other water-soluble polymers including: poly(acids), such as poly(acrylic acid), poly(methacrylic acid), poly(N-isoacrylimide), and poly(vinyl pyrrolidone); as well as co-polymers of these and other polymer moieties. In some embodiments, the conjugate is a microgel. In some embodiments, each microgel is linked to 5,000 or more (e.g., 6,000, 10,000, 15,000, or other numbers of similar magnitude) molecular beacon probes and corresponding amplification primers. In some embodiments, the microgel is 100-10,000 nm in diameter, (e.g., 100-1,000 nm in diameter). In some embodiments, (e.g., where the microgel is bound to a solid substrate), the hydrated microgel is 50-500 (e.g., 60-120) nm or more in height.

In some embodiments, when hydrated in an aqueous medium, the conjugate includes a transition region from the aqueous medium at the conjugate surface to a pure hydrated region within the conjugate in which the conjugate becomes gradually more crosslinked. In some embodiments, the transition region has a thickness at least equal to the combined length of the probe and the affinity pair or to the length of a ds-DNA bridge tethered at each end by the specific amplification primers, each with an affinity pair. In some such embodiments, the molecular beacon probes and the amplification primers are in the most water-like environment possible while remaining tethered to the gel. In such embodiments, the signal-to-background (SBR) ratio of the molecular beacon probe for the target can be 5 or greater, e.g., as high as 10, 20, 30 or greater. In some such embodiments, the SBR is 10% or greater (e.g. 20%, 30%, 40%) of a reference SBR ratio determined using a reference probe identical to the tethered molecular beacon probe, except that the reference probe is in a solution and not tethered.

In some embodiments, the conjugate described above can further involve a substrate to which the conjugate is attached. In some embodiments, the invention also provides an array containing (i) a support (the substrate) having a plurality of unique locations and (ii) a plurality of conjugates as described above for different targets, where each conjugate is immobilized to a unique location of the support corresponding to a target. Location on the support is one way in which the specific target associated with a particular conjugate can be identified. In some embodiments, the density of biospecific probes and corresponding primers at one of the plurality of unique locations is at least 1,000 pre, at least 10,000 pre, or at least 20,000 pre. In some embodiments, each microgel on a unique location has a hydrated height in the range of about 50 to about 500 nm, much higher density of probe molecules and corresponding primers projected onto the two-dimensional surface of the substrate/support as compared to probes tethered to the substrate by spacer molecules and/or tethering moieties that are short (about the same size as the probe/primer molecule itself). In some embodiments of the present invention, a microgel having a diameter of 400 nm and a height of 100 nm has about 11,800 probes per microgel, which is equivalent to a density of over 20,000 probes/μm² when referenced to the area of the substrate/support. In some embodiments, one or more primers are tethered to specific conjugates on the array, reducing restrictions on multiplexing due to primer-primer interactions such as dimerization, and, in some cases, entirely eliminating such restrictions.

In a third aspect of the present invention, the device, one or more conjugates, or an array described above can be used in a method for detecting the presence of one or more targets in a biological sample. Embodiments of methods according to the present invention include the steps of (a) providing a device, one or more conjugates, or an array described above; (b) contacting the device, the one or more conjugates, or the array with the biological sample for a period of time under conditions permitting binding between the one or more targets and the corresponding molecular beacon/hairpin probes or to the corresponding amplification primers; and (c) determining the presence of the binding between the one or more targets and the corresponding probes by fluorescence imaging thereby detecting the presence of the one or more targets. Because of the small size of the conjugate and the high density of the probes and primers, the method can be carried out for high-throughput detection of multiple targets in a sample simultaneously.

A fourth aspect of the present invention provides methods for making a conjugate described above. Methods according to the present invention include the steps of: (i) providing a gel that contains a first member of an affinity pair; (ii) contacting the gel with molecular beacon probes specific for a target, each molecular beacon probe comprising a second member of the affinity pair, for a period of time under conditions permitting binding between the first member and second member; (iii) removing molecular beacon probes that do not bind to the first member; (iv) contacting the gel with amplification primers that produce targets specific to the molecular beacon probes, each primer comprising another member of the affinity pair, for a period of time under conditions permitting binding between the first member and second member; and (v) removing primers that do not bind to the first member. The providing step can be carried out by a process having the steps of: (i) obtaining a substrate; (ii) depositing a precursor of the gel on the substrate a layer; and (iii) exposing the substrate and the layer of precursor to an electron radiation for a period of time under conditions permitting cross-linking within the gel precursor and cross-linking between the resulting gel and substrate.

A fifth aspect of the present invention provides methods for making arrays of the types discussed above. Such methods include the steps of: (i) obtaining a plurality of conjugates described above, which are specific for a plurality of targets, respectively; (ii) obtaining a support that has a plurality of unique locations; and (iii) depositing said plurality of conjugates on the plurality of unique locations, respectively.

### EXAMPLES

The description of the present invention includes a number of non-limiting examples (i.e., Examples 1-7) to illustrate various features of certain embodiments of the present invention, as presented below.

### Example 1

### Optimizing the Number and Distribution of Gel-Tethering Sites

Example 1 illustrates control of the concentration of tethering sites on and near the surface of a biotinylated PEG (B-PEG) microgel according to an embodiment of the present invention. In embodiments of the present invention, the spacing between adjacent tethered molecules may be controlled so as to maximize the amplification efficiency and/or maximize the signal from fluorescing molecular beacon probes.

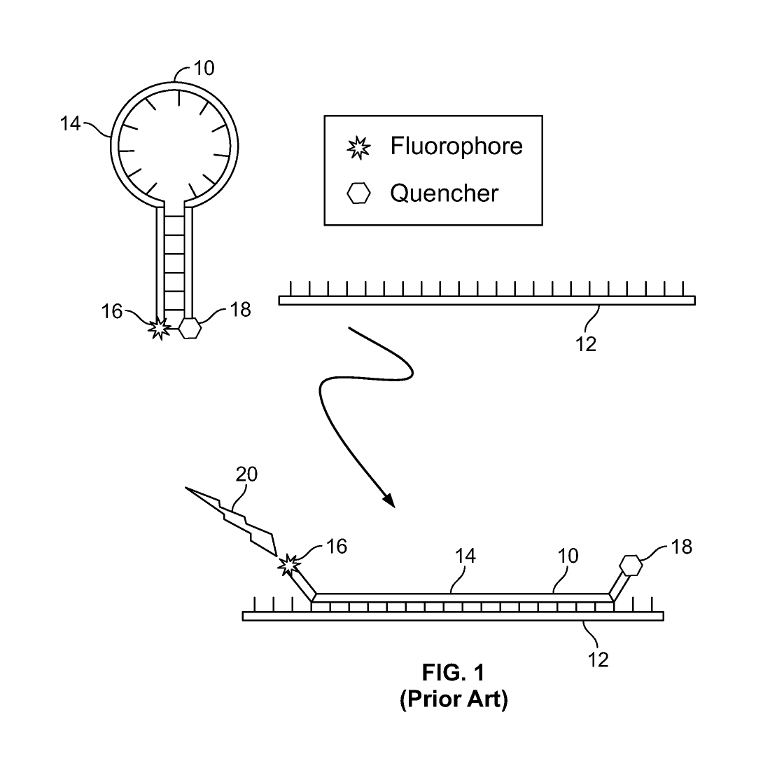

With respect to Example 1, FIG. 1 is a schematic illustration of a generalized molecular beacon probe 10 and its complementary target 12. Molecular beacon probes 10 are hairpin-shaped oligonucleotides that open when the loop section 14 binds in a complementary manner (i.e., is hybridized) to its complementary target 12. Hybridization separates the fluorophore 16 and quencher 18, so the molecular beacon fluoresces, emitting a photon 20. Nonspecific interactions of molecular beacon probes, such as probe 10, with solid surfaces can lead to background fluorescence in the absence of hybridization, which has hindered the use of such probes in microarrays. Conformational changes are required for such probes to hybridize to a complementary target and to open.

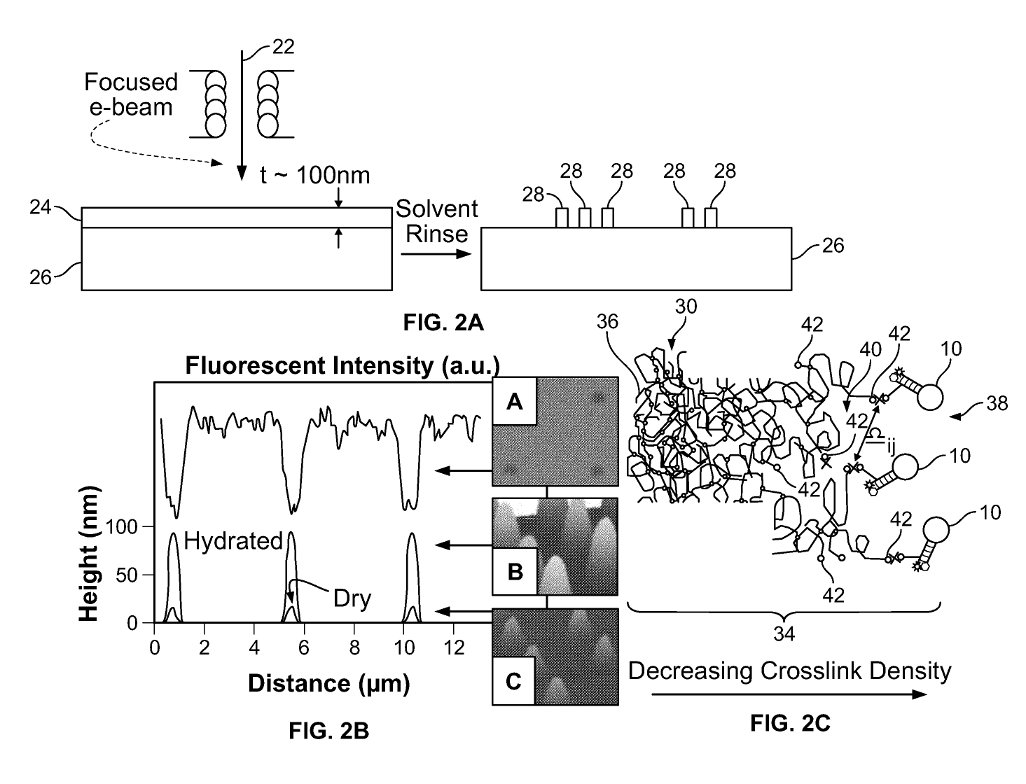

FIGS. 2A-2C constitute a schematic illustration of the synthesis and properties of surface-patterned microgels created by electron-beam patterning of biotinylated PEG thin films. Referring first to FIG. 2A, in an embodiment of the present invention, a focused electron beam 22 (e.g., 2 keV incident electron energy; 50 fC electron dose) crosslinks a biotinylated PEG thin film 24 on a substrate 26, and binds the resulting microgels 28 to the substrate surface. Unirradiated B-PEG is washed away using a good solvent, leaving behind individual microgels that are appropriately sized to serve as bases for tethered molecules. For example, such microgels may have dimensions of about 400 nm in diameter and 100 nm in height. Referring to FIG. 2B and the related microphotographs of Inset A, Inset B, and Inset C, the irradiation conditions can be chosen so that each microgel resists the non-specific adsorption of biomolecules such as proteins and oligonucleotides. Referring to the atomic force microscopic (AFM) image of Inset A, microgels 30 resist the non-specific adsorption of the exemplary protein laminin, as indicated by the absence of fluorescence. Referring to Inset B and Inset C, AFM images of hydrated (Inset B) and dry (Inset C) microgels show fluorescence of tethered beacon probes. Referring to FIG. 2C, the spatial distribution of energy deposited by a point exposure of the electron beam creates a highly non-uniform crosslink density in the polymer microgel 30, which that decreases asymptotically with increasing distance from the microgel center. This results in a transition region 34 from a hydrated region 36 within the microgel 30 to the aqueous medium 38 at the microgel surface 40 in the direction of decreasing crosslink density. Molecular beacon probes 10 and oligonucleotide primers (not shown) bound to the microgel surface 40 are thus in a highly hydrated, conformationally unconstrained, and exposed position. Optimizing the spacing δᵢⱼ between tethering points 42 is important for minimizing conformational constraints and for maximizing signal intensity.

In order to fluoresce, the gel-tethered molecular beacon probes 10 must hybridize and open. As discussed with respect to FIG. 1 and FIG. 2A, FIG. 2B, and FIG. 2C, both steps require conformational changes which can be inhibited by the close proximity of the molecular beacons 10 to each other. The relative proximity of adjacent beacons 10 or primers (not shown) can be adjusted by controlling the spatial distribution of the SA-activated biotin (SA-B) sites (such as sites 42) at microgel surfaces 40. Such sites 42 serve as anchoring points of the tethered probes 10. Too high a concentration causes conformational constraints. Too low a concentration reduces the available signal. Determining the concentration of SA-B sites at a microgel surface 40 that maximizes the signal from hybridized molecular beacons 10 is thus an important element of an integrated gel-tethered diagnostic platform. The balance between the number of tethered oligonucleotides and conformational constraints is even more important when both the amplification primers and the molecular beacon probes 10 are tethered to the microgels 30. In addition to the need for amplicons to hybridize with the beacons 10, complementary oligonucleotides and enzymes have to interact with the primers.

The concentration of biotins within a microgel 30 can be controlled. In the case of microgels formed by electron-beam patterning of PEG thin films, for example, the biotin concentration can be controlled by varying the molecular weight of the B-PEG precursor polymer or by blending B-PEG homopolymer with hydroxyl-terminated PEG homopolymer. Bifunctional biotinylated PEG homopolymer is commercially available with molecular weights ranging from 1 kDa-10 kDa, and monofunctional biotinylated PEG is available over an even greater range of molecular weights. Other functionalized polymeric precursors can be custom synthesized. To further control the inter-oligonucleotide spacing, one can either use homopolymer precursor of higher/lower molecular weight, which will affect the electron dose required for crosslinking, or by blending biotinylated PEG with hydroxyl-PEG of the same, or different, molecular weight. Alternatively, after microgel patterning, the concentration of SA-activated biotins actually functionalized can be controlled by varying the oligonucleotide concentration in the functionalizing solution or adjust the time allowed for these oligonucleotides to tether to the SA-activated biotin sites.

The average number of molecular beacon probes, or other tethered molecules, on a microgel 30 can be determined using an established streptavidin-release assay. As a substrate, one can use, for example, a 5 mm×7 mm Si substrate with microgels patterned on it in a square array with 1 μm inter-gel spacings. Such a surface thus contains enough microgels (e.g., on the order of 10⁶) to yield detectable and reliable signals, and, since the number of microgels on the surface is known, the average signal per microgel can be determined. One can, for example, expose such a surface to 500 μL 95% of formamide solution with 10 mM EDTA (pH 8.2) for 1 hr at 65° C. The SA-biotin bonds break because of this treatment and release fluorescently labeled reagents into solution, the concentration of which can be determined spectro-photometrically.

### Example 2

### Beacon and Primer Design for Gel Tethering

Example 2 provides an example of the modification of molecular beacons suitable for use untethered in aqueous solution in order to make them compatible with tethering to B-PEG microgels.

Much is already known about the design of molecular beacon probes for use in untethered assay applications. These can be modified, or new ones designed if necessary using established design techniques, to render them compatible with a gel-tethering approach. Similarly, many primers which create amplicons specific to corresponding probes have been identified or can be designed using established methods, and these, too, can be modified to render them compatible with a gel-tethering approach. The amplification primers are typically linear oligonucleotides. They can be synthesized with biotin, a spacer of oligo ethylene glycol (e.g. SP9), and an oligonucleotide spacer of controllable length (e.g., 10 T) at their 5′ end. Such molecules can be tethered to microgels via the SA-B interaction.

Table 1 summarizes molecular beacon probes, NASBA amplification primers, and complementary targets and templates appropriate for simplex or multiplex assays of bloodstream infection, according to an embodiment of the present invention, and is adapted from: Zhao et al. J. Clinical Microbiology, 2009. 47(7): pp. 2067-2078, the disclosure of which is incorporated by reference herein. The molecular beacon probes and NASBA amplification primers listed in Table 1, once modified with appropriate spacers and tethering moieties, are appropriate for use in applications involving gel-tethered amplification and/or gel-tethered detection according to embodiment of the present invention. Assays such as this one, or others which can be developed with appropriate design of molecular beacon probes and amplification primers, can be used in a gel-tethered integrated diagnostic platform according to an embodiment of the present invention. Embodiments of the present invention are also applicable to RNA/DNA amplification and detection applications, such as cancer detection or cancer identification to which gel-tethered molecular diagnostic approaches can be applied.

TABLE 1
Primers, probes, and complementary targets for model multiplexed infection assay
Modified from: Zhao, Y., S. Park, B. N. Kreiswirth, C. C. Ginocchio, R. Veyret, A. Laayoun, A.
Troesch, and D. S. Perlin, Rapid real-time nucleic acid sequence-based amplification-molecular
beacon platform to detect fungal and bacterial bloodstream infections. J. Clinical Microbiology,
2009. 47(7): pp. 2067-2078.
Pan Gram Positive Bacteria

| | |
|---|---|
| Forward primer (P2) | 5′-TACGGGAGGCAGCAGT-3′ |
| Reverse primer (P1) w/ T7 | 5′′-AATTCTAATACGACTCACTATAGGGGCTGCTGGCACGTAGT |
| | TAGCCGTGGCTTTC-3′ |
| 16S Pan-GrP MB | 5′-Alexa488-CGAGCTAGCAACGCCGCGTGAGTGAAGCTCG- |
| | BHQ2-Biotin-3′ |
| 16S Pan-GrP MB | 5′-GACCTTCATCACTCACGCGGCGTTGCTCCGTC-3′ |
| Synthetic Target | |
| DNA Template (+) | 5′-TACGGGAGGCAGCAGTAGGGAATCTTCCGCAATGGGCGAA |
| | AGCCTGACGGAGCAACGCCGCGTGAGTGATGAAGGTCTTCG |
| | GATCGTAAAACTCTGTTATTAGGGAAGAACATATGTGTAAGTA |
| | ACTGTGCACATCTTGACGGTACCTAATCAGAAAGCCACGGCT |
| | AACTACGTGCCAGCAGCCCC-3′ |

Pan Gram Negative Bacteria

| | |
|---|---|
| Forward primer (P2) | 5′-CCTGATGCAGCCATGCCGCGTG-3′ |
| Reverse primer (P1) w/ T7 | 5′-AATTCTAATACGACTCACTATAGGGCACGGAGTTAGCCGGT |
| | GCTT-3′ |
| 16S Pan-GrN MB | 5′ Alexa 488-CGAGCTTGAAGAAGGCCTTCGGGTTGTAAAGAG |
| | CTCG-BHQ2-Biotin-3′ |
| 16S Pan-GrN MB | 5′-TGAAAGTACTTTACAACCCGAAGGCCTTCTTCATACAC-3′ |
| Synthetic Target | |
| DNA Template (+) | 5′-CCTGATGCAGCCATGCCGCGTGTATGAAGAAGGCCTTCGG |
| | GTTGTAAAGTACTTTCAGCGGGGAGGAAGGGAGTAAGTTAAT |
| | ACCTTTGCTCATTGACGTTACCCGCAGAAGAAGCACCGGCTA |
| | ACTCCGTG-3′ |

Candida

| | |
|---|---|
| Forward primer (P2) | 5′-GGAATCCGCTAAGGAGTGTG-3′ |
| Reverse primer (P1) w/ T7 | 5′-AATTCTAATACGACTCACTATAGGGCCATCCATTTTCAGGG |
| | CTAGT-3′ |
| 16S Pan-Candida MB | 5′ Alexa 594-CGCGATTAACAACTCACCGGCCGAATATCGCG- |
| | BHQ2-Biotin-3′ |
| 16S Pan-Candida | 5′-GCTAGTTCATTCGGCCGGTGAGTTGTTACACAC-3′ |
| Synthetic Target | |
| DNA Template (+) | 5′-GGAATCCGCTAAGGAGTGTGTAACAACTCACCGGCCGAAT |
| | GAACTAGCCCTGAAAATGGATGG-3′ |

Aspergillus

| | |
|---|---|
| Forward primer (P2) | 5′-CAGCAGTTGGACATGGGTTA-3′ |
| Reverse primer (P1) w/ T7 | 5′-AATTCTAATACGACTCACTATAGGGGAGAATCCACATCCAG |
| | GTGC-3′ |
| 16S Pan-Aspergillus MB | 5′-Alexa 594-CGACCGGCATAGGGAAGTTCCGTTTGGTCG- |
| | BHQ2-Biotin-3′ |
| 16S Pan-Aspergillus | 5′-CGCCTTTCAAACGGAACTTCCCTATGCCTTAGG-3′ |
| Synthetic Target | |
| DNA Template (+) | 5′-CAGCAGTTGGACATGGGTTAGTCGATCCTAAGGCATAGGG |
| | AAGTTCCGTTTGAAAGGCGCCCTCGTGCGCCGTGTGCCGAA |
| | AGGGAAGCCGGTTAACATTCCGGCACCTGGATGTGGATTCTC- |
| | 3′ |

Fungi

| | |
|---|---|
| Forward primer (P2) | 5′-CGGCTCTTCCTATCATACCG-3′ |
| Reverse primer (P1) w/ T7 | 5′-AATTCTAATACGACTCACTATAGGGCTAAACCCAGCTCACG |
| | TTCC-3′ |
| 16S Pan-Fungi MB | 5′-Alexa 594-CGCGATATTCGGTAAGCGTTGGATTGATCGCG- |
| | BHQ2-Biotin-3′ |
| 16S Pan-Fungi MB | 5′-TGGGTGAACAATCCAACGCTTACCGAATTCTGC-3′ |
| Synthetic Target | |
| DNA Template (+) | 5′-CGGCTCTTCCTATCATACCGAAGCAGAATTCGGTAAGCGTT |
| | GGATTGTTCACCCACTAATAGGGAACGTGAGCTGGGTTTAG- |
| | 3′ |

* P1 and P2 are both immobilized on PEG-microgels at their 5′ ends with oligo(ethylene glycol) (e.g. SP9) and oligo(thymine) spacers. For example, in the case of Gram Positive Bacteria: Solid Phase-P1: 5′-Biotin-SP9-TTTTTTTTTTAATTCTAATACGACTCACTATAGGGGCTGCTGGCACGTAGTTAGCCGTGGCTTTC-3 (SEQ ID NO: 26) Solid Phase-P2: 5′-Biotin- SP9-TTTTTTTTTTTACGGGAGGCAGCAGT-3′ (SEQ ID NO: 27)

### Example 3

### Solution NASBA with Gel-Tethered Molecular Beacon Probes

Example 3 illustrates the case of solution NASBA coupled with gel-tethered molecular beacon probes according to an embodiment of the present invention. In the present invention, copies of antisense counterparts to the biological targets may be amplified in solution using the existing NASBA protocols and interact favorably with gel-tethered probes.

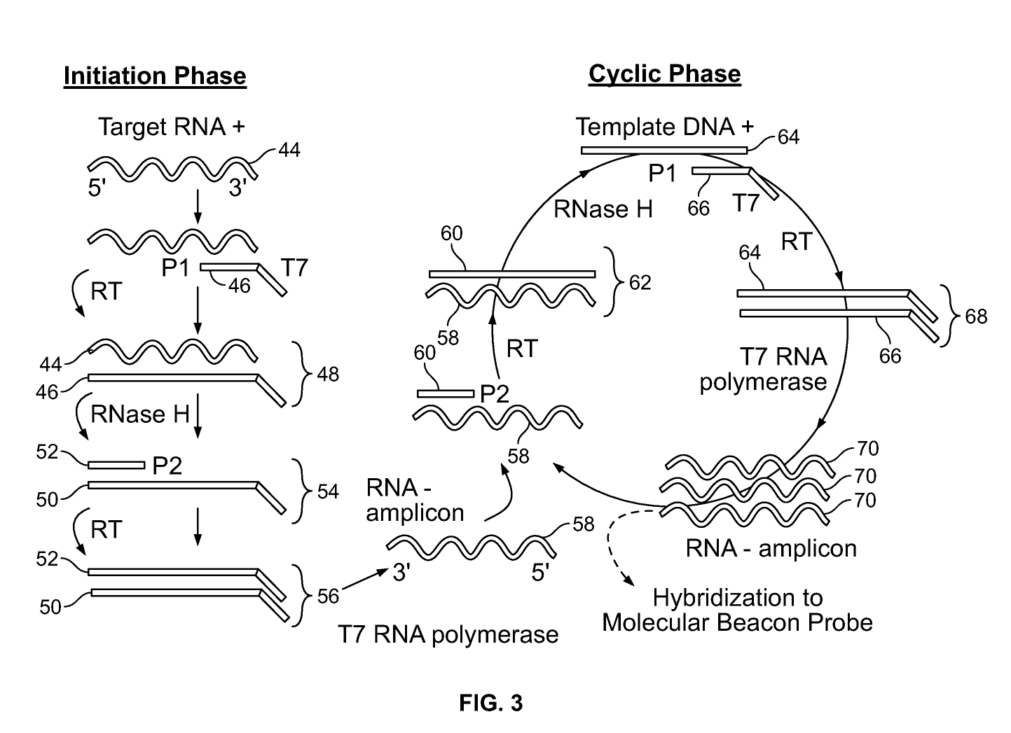

FIG. 3 is a schematic illustration of the NASBA amplification process. The entire amplification reaction can be separated into two phases: the initiation phase and the cyclic phase. An oligonucleotide (e.g., RNA+ 44) contains a biological target sequence (not shown). During the initiation phase, the RNA+ 44 hybridizes with the reverse primer P1 46 in solution. The hybridized P1 is then extended by AMV-RT (“RT”). The RNA+ strand 44 in the DNA/RNA+ hybrid I 48 is hydrolyzed by RNase H, leaving a complementary DNA (cDNA) 50. The cDNA hybridizes with P2 52 in solution. The hybridized P2 54 is then again extended by AMV-RT, forming a ds-DNA 56. Driven by T7 RNA polymerase, the ds-DNA structure 56 can generate multiple RNA− copies (i.e., “amplicons”) 58, antisense to RNA+ 44. Such RNA− amplicons are examples of complementary targets derived from a biological target. These RNA− amplicons 58 can either be detected when hybridizing with the molecular beacon probes (not shown) or join the cyclic phase. In the cyclic phase, P2 60 hybridizes with the RNA− 58 to form a DNA/RNA-hybrid 62, is extended by AMV-RT, and forms the DNA+ 64 after the hydrolysis reaction driven by RNase H. Subsequently, P1 66 hybridizes with the DNA+ 64, is extended by AMV-RT, and forms the ds-DNA 68, which can generate more RNA− 70 by T7 RNA polymerase.

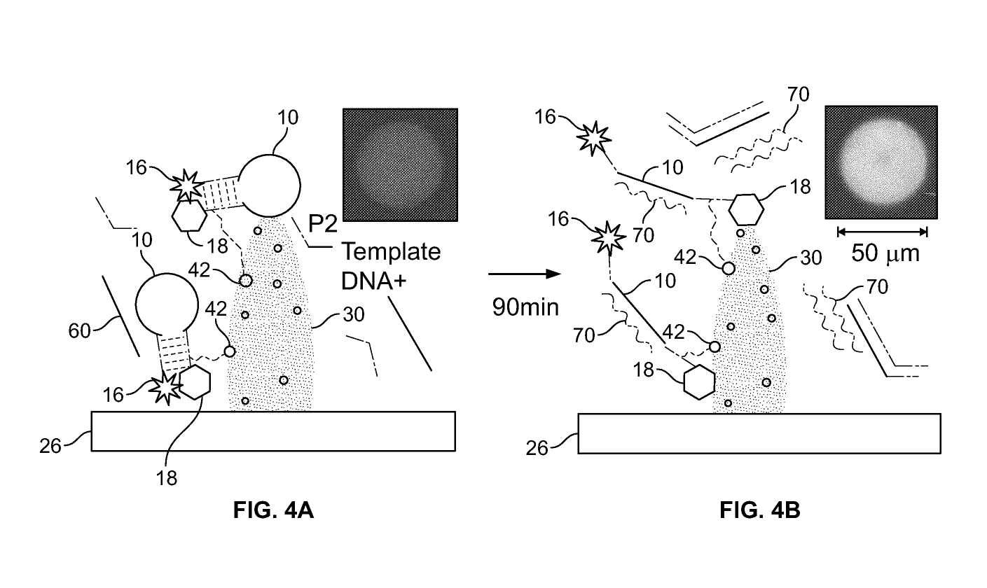

FIGS. 4A and 4B constitute a schematic illustration of the NASBA process in a solution which contacts microgel-tethered molecular beacon probes 10 in a microarray spot 30 (i.e., a microgel) on a solid substrate 26. In this process, RNA− amplicons 70 produced by the NASBA process can hybridize to molecular beacons 10. In this situation, primers, such as P1 66 and P2 60 are included in the solution surrounding the tethered molecular beacon probes 10. Therefore, when RNA+ (not shown) is present, the NASBA process occurs in solution and generates more and more RNA− amplicons 70. These RNA− amplicons 70 can reach the microgels 30 to which the molecular beacon probes 10 are tethered by diffusion through the solution. The hybridization between an RNA− 70 and a molecular beacon probe 10 changes the conformation of the molecular beacon probe 10 so that it fluoresces. FIGS. 4A and 4B illustrate the case of solution NASBA, where the primers, enzymes and reagents are free in solution. The inset micrographs show 50 μm diameter microgel spots functionalized with the molecular beacon probes 10. After 90 minutes exposure, the beacon-functionalized spot fluoresces brightly, as shown in FIG. 4B, as compared to the dark microphotograph of FIG. 4A wherein hybridization between the RNA− amplicons 70 and the probes 10 has not yet occurred.

In contrast to the vast majority of molecular diagnostics (MDx) tests that involve end-point labeling to induce fluorescence, the fluorescence emitted by microgel-tethered beacons increases continuously with time during the amplification process, realizing the concept of real-time detection.

### Example 4

### Partial Gel-Tethered NASBA with Gel-Tethered Molecular Beacon Probes

Example 4 illustrates the case of tethered molecular beacon probes together with one type of amplification primer being tethered to the same microgel, accordingly to an embodiment of the present invention. In the present invention, amplification and detection may occur when both a primer and a probe are gel-tethered.

Embodiments of the present invention provide a partial gel-tethered NASBA process. For example, biotinylated primer P1 66 and biotinylated molecular beacons 10 can both be tethered to the same microgel spots 30. This places the amplifying primer 66 and the detecting molecular beacon probe 10 in immediate proximity to each other. When template DNA+ 64, AMV-RT (not shown), and the T7 polymerase (not shown) are added to the surrounding solution, the NASBA process produces RNA− amplicons 70, which subsequently hybridize with the molecular beacon probes 10 causing them to fluoresce. While untethered amplification primers (not shown) can be included in the surrounding solution, these are not required for every embodiment of the present invention.

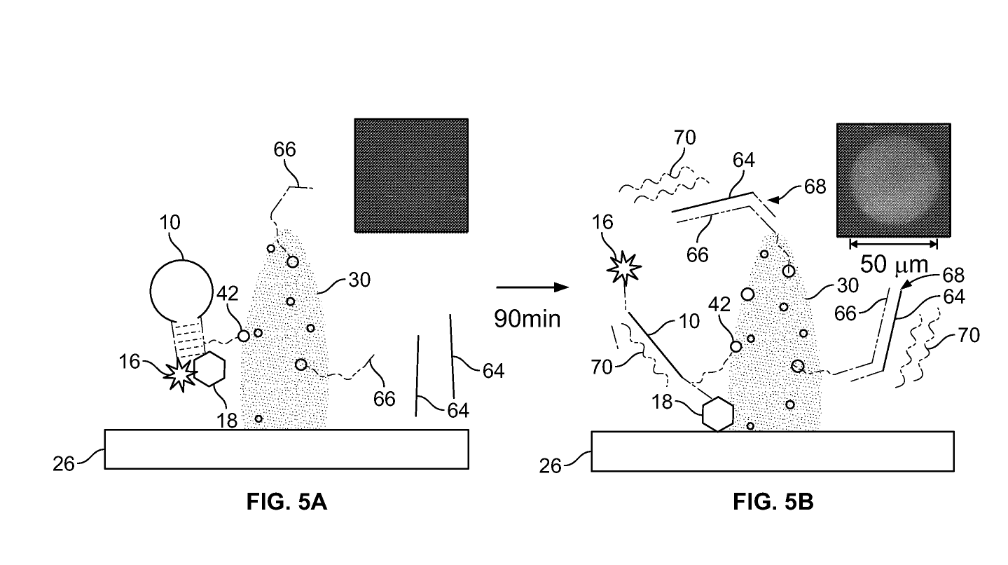

FIGS. 5A and 5B constitute a schematic illustration of a partial gel-tethered NASBA implementation with both primer P1 66 and the molecular beacon probes 10 micro-gel tethered to a microarray spot 30 and with the other reagents in the surrounding solution. DNA+ 64 in solution initiates the reaction. In the case where there are no primers in solution, the DNA+ 64 can only hybridize with the gel-tethered primer P1 66. The hybridized P1 66 is then extended by AMV-RT, forming a gel-tethered ds-DNA 68. When exposed to T7 RNA polymerase, this gel-tethered ds-DNA 68 then produces multiple copies of RNA− amplicons 70. Since both the ds-DNA 64 structures and the molecular beacon probes 10 are tethered on a microgel structure 30 in close proximity to each other, the diffusion distance for an RNA− amplicon 70 to reach a tethered molecular beacon probe is small, on the order of molecular length scales in many cases. The probability of amplicon hybridization to a tethered molecular beacon 10 is thus high. As a result of the conformation change associated with a hybridized molecular beacon probe 10, a fluorescent signal is generated (see the photomicrograph in FIG. 5B).

### Example 5

### Hybrid Partial Gel-Tethered and Solution NASBA with Gel-Tethered Molecular Beacon Probes

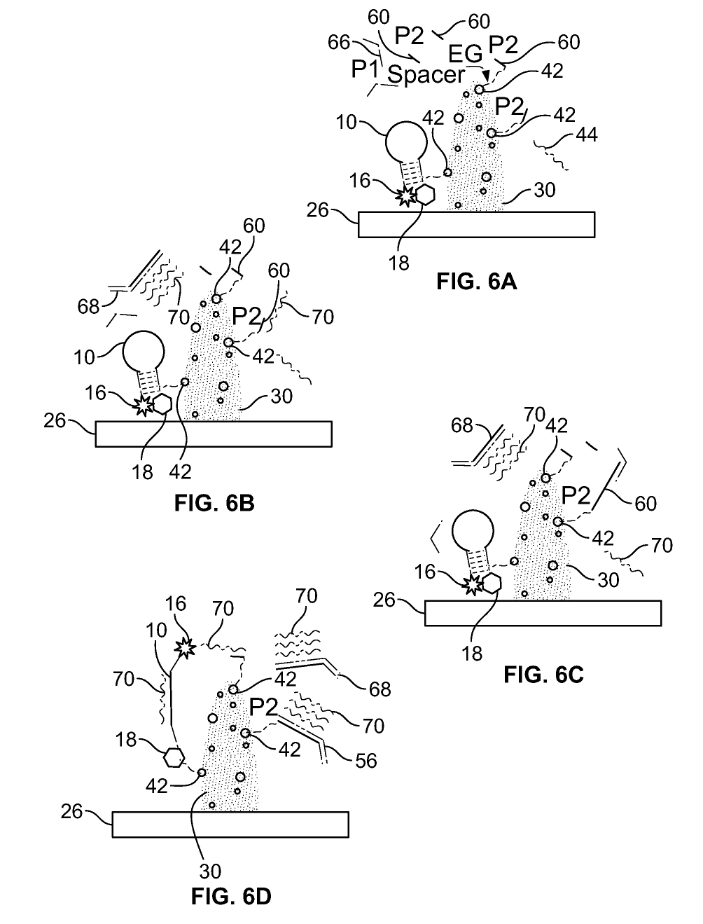

Example 5 illustrates the case of a hybrid amplification and detection process according to an embodiment of the present invention. FIGS. 6A-6D constitute a schematic illustration of such a hybrid process that includes amplification in the solution, amplification in conjunction with a gel-tethered primer, and detection by gel-tethered molecular beacon probes.

In the illustrated process, RNA+ 44 can be amplified using primers 60 in solution and using a primer 66 that is microgel tethered. This is a variation of combined amplification and detection, according to embodiments of the present invention, which involves a partial gel-tethered NASBA process together with a solution NASBA process. Such an embodiment can be used, for example, for the real-time detection of RNA. For example, biotinylated primer P2 60 and biotinylated molecular beacons 10 can both be tethered to the same microgel spots 30. This again places an amplifying primer 60 and the detecting molecular beacon probe 10 in immediate proximity to each other. When biological target RNA+ 44, AMV-RT (not shown), and the T7 polymerase (not shown) are added to the solution, the NASBA process produces RNA-amplicons 70, which can either subsequently hybridize with the beacons 10 causing them to fluoresce, or, hybridize with the gel-tethered P2 60. The hybridized, gel-tethered P2 60 is converted to a ds-DNA structure 68, tethered at one end, by exposure to and reaction with AMV-RT and RNase H. RNA− amplicons 70 generated by this ds-DNA structure 56 are in close proximity to the molecular beacon probes, accelerating the detection process.

### Example 6

### Gel-Tethered Double-Stranded DNA and Molecular Beacon Probes

Example 6 illustrates the case of RNA− amplicons created from double-stranded DNA that is tethered to a microgel spot. Such amplicons can hybridize to molecular beacon probes also tethered to that same spot.

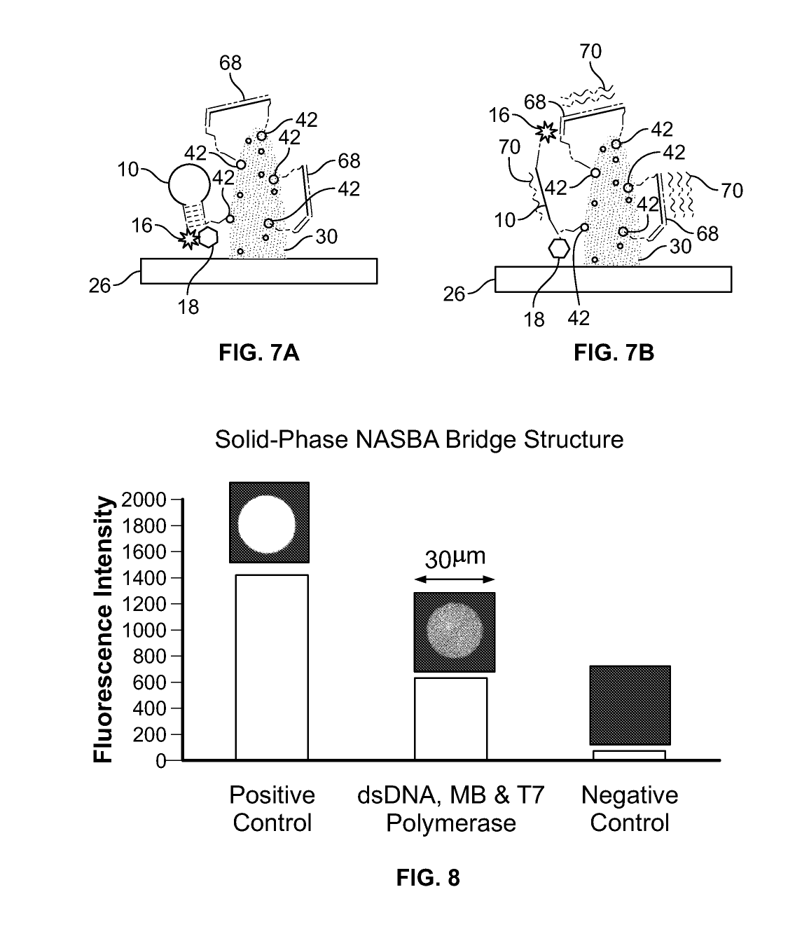

FIGS. 7A and 7B constitute a schematic illustration of gel-tethered double strand DNA (not shown) and gel-tethered molecular beacon probes 10 (FIG. 7A). Upon incubation in a buffer solution that includes T7 polymerase (not shown) and dNTP (not shown), RNA-amplicons 70 are generated, which hybridize with nearby molecular beacons 10, causing the molecular beacons to fluoresce 10 (FIG. 7B).

In this exemplary embodiment, double-stranded DNA with a biotin group at each end are created by mixing solutions containing two complementary single-stranded DNA molecules (not shown), each biotinylated at one end. After mixing, this solution is heated to 65° C. for 3 minutes to melt any hybridizations and then slowly cooled to room temperature during which time biotinylated ds-DNA forms 68. When exposed to T7 polymerase, RNA-amplicons 70 are created from the ds-DNA 68. Microgel spots can be functionalized with biotinylated ds-DNA 68 and biotinylated molecular beacon probes 10, which are complementary to the RNA− amplicons 70. For example, a solution of ds-DNA 68 and molecular beacon probes 10 can be exposed to streptavidin-activated biotinylated microgel spots 30. After about 60 minutes incubation, both ds-DNA 68 and molecular beacons 10 are tethered to the microgels 30 (see FIG. 7A). When subsequently exposed to a solution containing T7 polymerase and dNTP and held isothermally at an appropriate temperature (41° C.), RNA− amplicons 70 are created from the tethered ds-DNA 68 (see FIG. 7B). Since the concentration of RNA− amplicons 70 is highest near the microgel spots 30, there is a high probability that a fraction of these will bind to complementary molecular beacon probes 10 tethered to that same microgel spot 30 (see FIG. 7B).

FIG. 7A, discussed above, illustrates the ds-DNA 68 bridging between two different binding sites 42 on an individual surface-patterned microgel 30 together with a closed molecular-beacon probe 10 tethered to that same microgel 30. FIG. 8, discussed below, describes the situation after gel-tethered molecular beacons 10 hybridize with RNA− amplicons 70 generated from the ds-DNA 68.

FIG. 8 is a bar chart of the average fluorescence intensity emitted by three different microgel spots, each associated with an AFM image of the microgel spot being reported. The column labeled positive control shows the average fluorescence intensity after immobilized molecular beacons hybridize with 0.5 μM synthetic targets in solution. In this case, no amplification occurs. The column labeled dsDNA, MB & T7 polymerase shows the fluorescence intensity after a microgel spot functionalized with tethered molecular beacon probes was incubated for 90 minutes in NASBA reaction buffer containing T7 polymerase but no other enzymes. The column labeled negative control shows the fluorescence intensity measured when there are no targets, either amplified or spiked, present that are complementary to the tethered molecular beacon probes.

The microgel spots were each functionalized with both ds-DNA and molecular-beacon probes. The positive control microgel spot was exposed to a NASBA amplification solution that contained no enzymes, but was spiked with ss-DNA complementary to the gel-tethered molecular beacon probes. In this case, the ss-DNA hybridizes to the molecular-beacon probes. FIG. 8 shows that this case generates a high fluorescence signal. The negative control microgel spot was exposed to a NASBA solution containing no enzymes and no targets complementary to the molecular beacon probes. In this case there is no hybridization, and the fluorescence intensity measured is low. The T7 microgel spot was exposed to NASBA solution containing no enzymes other than T7 polymerase. In this case, FIG. 8 shows that an intermediate level of fluorescence intensity is observed indicating that RNA− was both successfully amplified from the tethered ds-DNA and then hybridized to the tethered molecular beacon probes. When other enzymes such as RNase H are present in the NASBA reaction solution, fluorescence from the microgel spot can still be observed, albeit at a different intensity than in examples where RNase H is absent.

### Example 7

### Gel-Tethered NASBA Amplification Primers and Molecular Beacon Probes

Example 7 illustrates the case of two amplification primers and one corresponding molecular beacon probe tethered to the same microgel, according to an embodiment of the present invention. In the present invention, gel-tethered NASBA amplification may be coupled with gel-tethered molecular beacon detection.

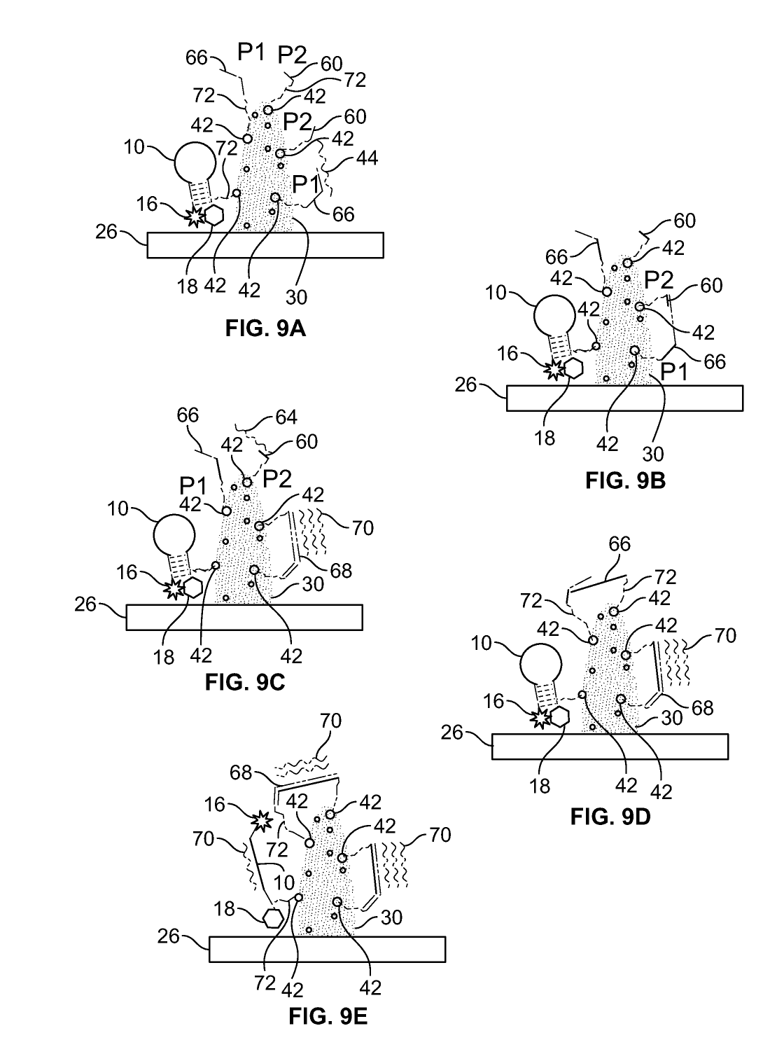

Such embodiments of the present invention provide arrayed microgel spots which enable the capture, amplification, and detection of biospecific oligonucleotide targets. FIGS. 9A-9E constitute a schematic illustration of these processes. The molecular beacon probe(s) 10 and the amplification primers (P1 66 and P2 60) are tethered to a microgel 30 via streptavidin-biotin (SA-B) binding 42. Primer P1 66 contains a T7 promoter. Both P1 66 and P2 60 can contain an ethylene glycol 72 (EG) spacer of controllable length. Biological target RNA+44, which can, for example, be isolated from a clinical sample, binds to P1 66 (see FIG. 9A) and, with AMV-RT, is extended to form c-DNA 64 with a complementary sequence (see FIG. 9B). RNase H exposure removes the RNA+ 44, so the 3′ end of the c-DNA 64, created by extending the gel-tethered P1 66, can then bind to a tethered P2 60. Exposure to AMV-RT forms the tethered ds-DNA bridge 68 and, with T7 RNA polymerase, RNA− amplicon 70 is repeatedly produced from the tethered ds-DNA template 68 (see FIG. 9C). Importantly, the ds-DNA bridge 68 is tethered, and it becomes a continuous point source for RNA− amplicon 70 production. The RNA− 70 can diffuse away, bind to a nearby molecular beacon 10, or bind to a tethered P2 60 (the latter case is illustrated by FIG. 9D). In the latter case, AMV-RT and RNase H produce another ds-DNA bridge 68, which again remains tethered. This bridge 68 also produces RNA− amplicon 70. When RNA− 70 binds to a molecular beacon 10, the fluorophore 16 and quencher 18 are separated and the beacon 10 fluoresces (see FIG. 9E). Significantly, the ds-DNA 68 is tethered in close proximity to other tethered primers 60, 66 and to tethered molecular beacon probes 10. This close proximity minimizes the distance over which the RNA− 30 must diffuse in order to reach a complementary target, thus enhancing the efficiency of the amplification and detection process.

When multiple spots 30 such as these are arrayed on a surface of, for example, glass or silicon, according to embodiments of the present invention, each spot 30 may be functionalized with a set of molecular beacon probes 10 and corresponding amplification primers 60, 66 specific to a particular target 44, 70 (i.e., the various microgels 30 in an array may be modified such that each modified microgel 30 becomes specific to a different target). The number of biological or complementary targets 44, 70 that may be probed by the array thus depends on the number of spots 30 in the array. Since the amplification primers are all tethered to microgels within each spot, this embodiment of the invention eliminates the need to have primers in solution. Consequently, the complexation of primers free in solution is eliminated. An array according to this embodiment of the present invention can thus become highly multiplexed while still enabling all of the spots in the array to be exposed to the same, single solution that will contain one or more targets, and eliminates the need to create individual reaction chambers, each of which contains only one primer set and the corresponding probe and all of which contain a sample of the parent target-containing solution.

Further to the exemplary embodiments presented above, the present invention further includes, but is not necessarily limited to, the following embodiments, each of which operates according to the processes discussed above.

Embodiments of the present invention provide a device for capturing, amplifying, detecting, and identifying one or more biological target molecules. In some embodiments, the device comprises an array of isolated areas on a surface, each area having quenched probes and amplification primers tethered thereto. In some embodiments, each of the aforesaid probes and primers is tethered to a structure that is patterned on one of the isolated areas. In some embodiments, each of the isolated areas includes one or more patterned microgels. In some embodiments, the aforesaid probes and primers are tethered to the structure through one or more tethering moieties that are chemically or physically incorporated into the structure. In such embodiments, the aforesaid probes are molecules that undergo a conformational change when they bind to a complementary molecule (i.e., a complementary target), such that the conformational change leads to a detectable signal. In some embodiments, the structure is hydrophilic and, except for the tethering moieties, interacts weakly with the probes, the amplification primers, the enzymes used for amplification, the complementary targets, and the probe-tethering molecules, such that the structure does not interfere with construction or use of the device. In some embodiments, the structure, when hydrated, provides a gradient in crosslink density which is lowest at the surface of the structure, and is such that, at some position in the structure, the target molecule is unable to penetrate further towards the center of the structure. In some embodiments, the complementary target is uniquely identified with the biological target.

In some embodiments of the device, the array contains at least five areas each containing a structure tethered to a set of probes and primers specific to a particular complementary and/or biological target molecule. In some embodiments of the device, the array contains at least ten areas each containing a structure tethered to a set of probes and primers specific to a particular target molecule. In some embodiments of the device, the array contains at least twenty areas each containing a structure tethered to a set of probes and primers specific to a particular target molecule. In some embodiments of the device, the array contains more than twenty areas each containing a structure tethered to a set of probes and primers specific to a particular target molecule. In some embodiments of the device, each of the sets of probes and primers in at least some of the areas corresponds to a different target molecule, and all of the areas are simultaneously exposed to a primer-free aqueous solution to test whether or not that solution contains any of the target molecules corresponding to any of the areas within the array.

In some embodiments of the device, the structure is a patterned microgel. In some such devices, the structure is an electron-beam-patterned microgel. In some such devices, the microgel includes poly(ethylene) glycol. In some such devices, the structure is a biotinylated, e-beam patterned poly(ethylene) microgel.

In some embodiments of the device, the tethered probes are hairpin oligonucleotides labeled with a fluorophore and a non-fluorescent quencher, the combination of which fluoresces when the hairpin binds to its complementary target. In some embodiments, the tethered primer or tethered primers are oligonucleotides, which can create targets complementary to the probes by a process including an operation on the biological target molecule. In some embodiments of the device, the biological target molecule includes an oligonucleotide. In some embodiments, the oligonucleotide includes non-natural nucleotides, nucleotide analogs, or non-natural inter-nucleotide linkages.

In some embodiments of the device, the tethered probes include linear DNA, and are not necessarily in a hairpin configuration. The linear DNA is arranged such that it hybridizes to the complementary target. The hybridized linear DNA is detected using a post-hybridization fluorescent labeling method, examples of which are known in the art.

Some embodiments of the device include a tethering moiety based on the streptavidin-biotin interaction.

In some embodiments of the device, the probes are such that, when untethered in solution and bound to their complementary targets, they have a signal-to-background ratio that is not more than ten times as large as the signal-to-background ratio that they have in the device. In some embodiments of the device, the probes are such that, when untethered in solution and bound to their complementary targets, they have a signal-to-background ratio that is not more than five times as large as the signal-to-background ratio that they have in the device.

Embodiments of the present invention further include methods of making the aforesaid devices of the present invention, as enabled by the present disclosure, including the disclosures of the documents incorporated by reference herein. Embodiments of the present invention further include methods of capturing, amplifying, detecting, and/or identifying one or more target biological molecules using the aforesaid devices of the present invention, as enabled by the present disclosure, including the disclosures of the documents incorporated by reference herein.

It should be understood that the embodiments described herein are merely exemplary in nature and that a person skilled in the art may make many variations and modifications thereto without departing from the scope and spirit of the present invention. All such variations and modifications, including those discussed above, are intended to be included within the scope and spirit of the invention, as defined by the appended claims.

We claim:

1. A device for detecting at least one target molecule derived from at least one biological source, comprising:
a hydrophilic structure adhering to a surface and having a first plurality of first tethering moieties incorporated therein;
a plurality of quenched molecular beacon probes and a plurality of amplification primer molecules, each of said probes tethered to said hydrophilic structure by a first tethering molecule interacting with one of said tethering moieties, each of said primer molecules tethered to said hydrophilic structure by means of a second tethering molecule interacting with another of said tethering moieties, wherein each of said probes is a molecule that binds to a molecule that is complementary to said probes along the complementary portions of said probes and said complementary molecules in such a manner that said probe undergoes a conformational change that causes said probe to fluoresce, wherein the complementary molecules are uniquely identified with the at least one target molecule, and wherein said probes and said primer molecules interact not more than weakly with said hydrophilic structure.

2. The device according to claim 1, wherein said hydrophilic structure includes a cross-linked polymer arranged such that said hydrophilic structure in a hydrated state contains a gradient in cross-link density which increases from an outer surface of said hydrophilic structure to an interior portion of said hydrophilic structure such that at some position along said gradient the at least one target molecule and the complementary molecule are unable to penetrate further into said interior portion of said hydrophilic structure.

3. The device according to claim 1 wherein each of said probes includes a first oligonucleotide that is a hairpin oligonucleotide labeled with a fluorophore and a non-fluorescent quencher, at least a portion of said hairpin oligonucleotide having a sequence of nucleotides that binds to at least a portion of the complementary molecule in a complementary manner.

4. The device according to claim 3 wherein the at least one target molecule includes a second oligonucleotide.

5. The device according to claim 3 wherein said primer molecules include third oligonucleotides which can be manipulated to create the complementary molecule by a process that uses the at least one target molecule as a template, the complementary molecule being a fourth oligonucleotide.

6. The device of claim 2, wherein said hydrophilic structure includes a microgel.

7. The device according to claim 6, wherein said hydrophilic structure is an electron-beam-patterned microgel.

8. The device according to claim 7, wherein said microgel includes poly(ethylene) glycol.

9. The device according to claim 1, wherein said tethering moieties interact with said first and second tethering molecules by a streptavadin-biotin interaction.

10. The device according to claim 3, wherein said first oligonucleotide includes one or more of a non-natural nucleotide, a nucleotide analog, or a non-natural inter-nucleotide linkage.

11. The device according to claim 1, wherein said probes are of a type that fluoresce when bound to the complementary molecule in an untethered state in an aqueous solution, the fluorescence having a signal-to-background ratio that is not more than five times as large as the signal-to-background ratio said probes have in said device.

12. The device according to claim 1, wherein said probes are of a type that fluoresce when bound to the complementary molecule in an untethered state in an aqueous solution, the fluorescence having a signal-to-background ratio that is not more than ten times as large as the signal-to-background ratio that said probes have in said device.

13. The device according to claim 1, wherein the target molecule is specific to its biological source.

14. The device according to claim 1, wherein said hydrophilic structure is a biotinylated, e-beam patterned poly(ethylene) microgel.

15. The device according to claim 1, wherein said tethered probes and said tethered primers are spaced apart by an average distance that allows a maximum number of said probes and said primers to be tethered to said hydrophilic structure without substantially constraining the conformational changes of said tethered probes.

16. The device according to claim 1, further comprising at least another hydrophilic structure adhering to said surface and having another plurality of tethering moieties incorporated therein, and another plurality of quenched molecular beacon probes and another plurality of second amplification primer molecules, each of said another probes tethered to said hydrophilic structure by a third tethering molecule interacting with one of said another tethering moieties, each of said another primer molecules tethered to said at least another hydrophilic structure by means of a fourth tethering molecule interacting with another one of said another tethering moieties, wherein each of said another probes is a molecule that binds to another complementary molecule that is complementary to said another probes such that said another probe undergoes a conformational change that causes said another probe to fluoresce, wherein said another probes and said another primer molecules interact not more than weakly with said another hydrophilic structure, wherein the another complementary molecules are uniquely identified with at least one different target molecule having a different chemical structure than the chemical structure of the at least one target molecule of claim 1, and wherein said another hydrophilic structure is spaced away from said hydrophilic structure of claim 1 and discontinuous therewith.

17. A device, comprising:
a plurality of hydrophilic structures, each of said hydrophilic structures having a respective plurality of molecular beacon probes and a respective plurality of amplification primer molecules tethered thereto, wherein each of said respective plurality of probes is arranged to bind in a complementary manner to a respective one of a plurality molecules that are complementary to said respective plurality of probes such that said each of said respective plurality of probes fluoresces when bound to its respective one of the respective plurality of complementary molecules, wherein each of said respective pluralities of complementary molecules is uniquely identified with a respective target molecule of a plurality of target molecules from one or more biological sources, the respective target molecule having a chemical structural that is distinct from the chemical structures of the plurality of other target molecules.

18. A system for detecting a plurality of target molecules from biological sources, comprising:
a plurality of hydrophilic structures, each of said hydrophilic structures having a respective plurality of molecular beacon probes and a respective plurality of amplification primer molecules tethered thereto, wherein each of said respective plurality of probes is arranged to bind in a complementary manner to a respective one of a plurality molecules that are complementary to said respective plurality of probes such that said each of said respective plurality of probes fluoresces when bound to its respective one of the respective plurality of complementary molecules, wherein each of said respective pluralities of complementary molecules is uniquely identified with a respective target molecule of the plurality of target molecules, the respective target molecule having a chemical structural that is distinct from the chemical structures of the plurality of other target molecules; and
an aqueous solution including the plurality of target molecules and a plurality of enzymes that cooperate with each said respective pluralities of amplification primer molecules to create a plurality of copies of the respective complementary molecules by a process that operates on the plurality of target molecules, wherein said plurality of hydrophilic structures are in contact with said solution such that said solution maintains said plurality of hydrophilic structures in a hydrated state.

19. The system of claim 18, wherein the plurality of target molecules includes a plurality of oligonucleotides, and said plurality of enzymes and said respective pluralities of primer molecules are arranged to implement a NASBA amplification process operating on the plurality of oligonucleotides.

20. The system of claim 19, wherein the plurality of oligonucleotides includes a plurality of RNA oligonucleotides.
